(19) United States
(12) Patent Application Publication
(10) Pub. No.: US 2022/0202654 A1
(43) Pub. Date: Jun. 30, 2022

(54) Enteral Feeding Tube with Polygonal Configuration

(71) Applicant: MEDELA HOLDING AG, Baar (CH)

(72) Inventors: David Y. Cho, Arlington Heights, IL (US); Shane Einloth, McHenry, IL (US)

(21) Appl. No.: 17/538,591

(22) Filed: Nov. 30, 2021

Related U.S. Application Data
(60) Provisional application No. 63/131,945, filed on Dec. 30, 2020.

(51) Int. Cl. A61J 15/00; A61F 7/00

(52) U.S. Cl. CPC A61J 15/0026; A61F 7/0085; A61J 15/0011

(57) ABSTRACT

A tube received, at least in part, by a breastmilk warmer includes a first segment having a first thermal conductivity, a second segment having a second thermal conductivity different from the first thermal conductivity, and a third segment having a third thermal conductivity different from at least one of the first and second thermal conductivities. The first segment is configured to be coupled to a source of fluid. The second segment is fluidly coupled to the first segment and disposed in a labyrinthine manner within a breastmilk warmer. The third segment is fluidly coupled to the second segment and is configured to be coupled to a feeding apparatus. The tube also includes a fluid lumen extending from the first segment through the second segment and to the third segment. The fluid lumen has a uniform diameter from the first segment to the third segment.

### CROSS-REFERENCE TO RELATED APPLICATION

This application claims priority under 35 U.S.C. § 119 to U.S. Provisional Application No. 63/131,945 filed Dec. 30, 2020, which is incorporated herein by reference in its entirety.

### FIELD OF THE DISCLOSURE

The disclosure relates generally to devices and methods to warm liquids. In particular, the disclosure relates to devices and methods used to more efficiently and effectively warm fluids used during enteral feeding.

### BACKGROUND

When an infant is born prematurely, with health problems, or endures a difficult birth, it is likely that the infant will be placed in a neonatal intensive care unit (“NICU”) for monitoring, care, and treatment, typically within twenty-four hours of birth. The duration of stay in the NICU depends on such factors as the severity and type of the infant's condition and the infant's ability to thrive and consume nourishment without the aid of a feeding tube. In a NICU environment, infants can be placed in or interact with several different kinds of equipment. For example, the NICU can include infant warmers, incubators, phototherapy equipment, monitors such as chest leads, pulse oximetry, a temperature probe, and blood pressure monitor, feeding tubes, intravenous catheters (IVs), central lines, arterial lines, ventilators, oxygen hood, and nasal cannulas.

When an infant is in need of nutrients but cannot consume food for various reasons, enteral feeding can be used to supplement the nutrients missing from the infant's diet. Enteral feeding refers to the intake of food via the gastrointestinal (“GI”) tract, which consists of the mouth, the esophagus, the stomach, and the intestines. In settings such as the NICU, enteral feeding often involves the use of feeding via a tube such as, for example, a nasogastric tube (NGT), an orogastric tube (OGT), a nasoenteric tube, an oroentric tube, a gastrostomy tube, and a jejunostomy tube. In some uses of the various enteral feeding tubes, the fluid passing through the tubing may need to be cooled or heated depending on the fluid passing therethrough.

### SUMMARY OF THE DISCLOSURE

A tube is configured to be received, at least in part, by a breastmilk warmer. The tube includes a first segment, a second segment, and a third segment. The first segment has a first thermal conductivity and is configured to be coupled to a source of fluid. The second segment has a second thermal conductivity that is different from the first thermal conductivity. The second segment is also fluidly coupled to the first segment and configured to be disposed in a labyrinthine manner within a breastmilk warmer. The third segment has a third thermal conductivity different from at least one of the first and second thermal conductivities. The third segment is also fluidly coupled to the second segment and configured to be coupled to a feeding apparatus. The first aspect also includes a fluid lumen that extends from the first segment through the second segment and to the third segment. The fluid lumen has a uniform diameter from the first segment to the third segment.

The second thermal conductivity of the second segment is preferably greater than the first thermal conductivity of the first segment and the third thermal conductivity of the third segment. While the percent of increase of the second thermal conductivity over the first thermal conductivity and the third thermal conductivity can vary, the second thermal conductivity is preferably at least five percent, fifteen percent, twenty-five percent, or thirty-five percent greater than the thermal conductivities of the first and third segments.

The first segment has a circular cross-section, the second segment has a polygonal cross-section, and the third segment has a circular cross-section.

A cross-section of the first segment has a first shape, a cross-section of the second segment has a second shape, and a cross-section of the third segment has a third shape, the second shape being different than at least one of the first shape and the third shape.

The second shape may be triangular and at least one of the first shape and the third shape is a circle.

Alternatively, the second shape may be rectangular, such as a square, and at least one of the first shape and the third shape is a circle.

In another alternative, the second shape may be hexagonal and at least one of the first shape and the third shape is a circle.

A tube is configured to be received, at least in part, by a breastmilk warmer and includes a first segment, a second segment, and a third segment. The first segment has a first outer surface having a first shape and is configured to be coupled to a source of fluid. The second segment has a second outer surface having a second shape and the second segment is configured to be disposed in a labyrinthine manner within a breastmilk warmer. The third segment is fluidly coupled to the second segment and configured to be coupled to a feeding apparatus. The second aspect also includes a fluid lumen extending from the first segment through the second segment and to the third segment. The fluid lumen has a uniform diameter from the first segment to the third segment. The second shape increases a thermal conductivity of the second segment relative to a thermal conductivity of the first segment and a thermal conductivity of the third segment.

The second shape of the second segment preferably increases the thermal conductivity of the second segment relative to the thermal conductivity of the first segment and the thermal conductivity of the third segment. While the percent of increase of the second shape of the second segment over the first shape of the first segment and the third shape of the third segment can vary, the second shape of the second segment preferably increases the thermal conductivity by at least five percent, fifteen percent, twenty-five percent, or thirty-five percent more than the first shape of the first segment and the third shape of the third segment.

The first outer surface has a circular cross-section, the second outer surface has a polygonal cross-section, and the third segment has a third outer surface that has a circular cross-section.

Alternatively, the second outer surface may be a triangle and at least one of the first outer surface and a third outer surface of the third segment is a circle.

In another alternative, the second outer surface may be a square and at least one of the first outer surface and a third outer surface of the third segment is a circle.

In yet another alternative, the second outer surface may be a hexagon and at least one of the first outer surface and a third outer surface of the third segment is a circle.

A breastmilk warming system of the present disclosure includes a breastmilk warmer and a tube that is coupled to a source of fluid and a feeding apparatus. The breastmilk warmer includes a housing, a labyrinthine channel disposed within the housing; and a warming assembly that is in communication with the labyrinthine channel and increases a temperature of the labyrinthine channel, thereby heating a segment of tubing received therein, which serves to warm fluid carried by the segment of tubing. The tube includes a first segment, a second segment, a third segment, and a fluid lumen. The first segment has a first thermal conductivity. The second segment has a second thermal conductivity different from the first thermal conductivity. The second segment is fluidly coupled to the first segment and is received in the labyrinthine channel of the breastmilk warmer. The third segment has a third thermal conductivity different from at least one of the first and second thermal conductivities. The third segment is fluidly coupled to the second segment. The fluid lumen extends from the first segment through the second segment and to the third segment. The fluid lumen also has a uniform diameter from the first segment to the third segment.

The second thermal conductivity of the second segment is preferably greater than the first thermal conductivity of the first segment and the third thermal conductivity of the third segment. While the percent of increase of the second thermal conductivity over the first thermal conductivity and the third thermal conductivity can vary, the second thermal conductivity is preferably at least five percent, fifteen percent, twenty-five percent, or thirty-five percent greater than the thermal conductivities of the first and third segments.

The first segment has a circular cross-section, the second segment has a polygonal cross-section, and the third segment has a circular cross-section.

The labyrinthine channel may have a polygonal cross-section that is substantially similar to the polygonal cross-section of the second segment.

A first cross-section of the first segment has a first shape, a second cross-section of the second segment has a second shape, and a third cross-section of the third segment has a third shape, the second shape being different than at least one of the first shape and the third shape.

Alternatively, the second shape of the second cross-section may be a triangle and at least one of the first shape of the first cross-section and the third shape of the third cross-section is a circle.

Alternatively, the labyrinthine channel of the breastmilk warmer may have a triangular cross-section that receives the second cross-section of the second shape of the second segment.

In another alternative, the second shape of the second cross-section may be a square and at least one of the first shape of the first cross-section and the third shape of the third cross-section is a circle.

In yet another alternative, the labyrinthine channel of the breastmilk warmer may have a square cross-section that receives the second-cross section of the second shape of the second segment.

In yet another alternative, the second shape of the second cross-section may be a hexagon and at least one of the first shape of the first cross-section and the third shape of the third cross-section is a circle.

In yet another alternative, the labyrinthine channel of the breastmilk warmer may have a hexagonal cross-section that receives the second-cross section of the second shape of the second segment.

A method of warming breastmilk includes providing a warmer including a housing, a labyrinthine channel disposed within the housing, and a warming assembly in communication with and configured to increase a temperature of the labyrinthine channel. The method further includes coupling a first segment of a tube to a source of fluid. The tube has a second segment fluidly coupled to the first segment and a third segment fluidly coupled to the second segment. The method additionally includes disposing the second segment of the tube in the labyrinthine channel of the warmer, and activating the warming assembly thereby increasing a temperature of the labyrinthine channel such that a temperature of a fluid from the source of fluid flowing through the second segment increases.

In disposing the second segment of the tube in the labyrinthine channel, the second segment preferably has a thermal conductivity greater than a thermal conductivity of the first segment and a thermal conductivity of the third segment. While the percent of increase of the thermal conductivity of the second segment relative to the thermal conductivity of the first segment and the thermal conductivity of the third segment can vary, the thermal conductivity of the second segment is preferably at least five percent, fifteen percent, twenty-five percent, or thirty-five percent greater than the thermal conductivity of the first segment and the thermal conductivity of the third segment.

In disposing the second segment of the tube in the labyrinthine channel, the second segment may have a polygonal cross-section, the first segment has a circular cross-section, and the third segment has a circular cross-section.

In disposing the second segment of the tube in the labyrinthine channel, a cross-section of the labyrinthine channel of the breastmilk warmer receives a cross-section of the second segment of the tube.

A method of assembling a breastmilk warming apparatus includes providing a tube and a breastmilk warmer having a housing, a labyrinthine channel disposed within the housing, and a warming assembly in communication with the labyrinthine channel and configured to increase a temperature of the labyrinthine channel. The method also includes coupling a first segment of the tube to a source of fluid, and coupling a second segment of the tube to the first segment of the tube. The second segment has a second thermal conductivity. The method further includes coupling a third segment of the tube to the second segment. The third segment is coupled to a feeding apparatus. The method additionally includes disposing the second segment of the tube in the labyrinthine channel of the breastmilk warmer.

In disposing the second segment of the tube in the labyrinthine channel, the second segment preferably has a thermal conductivity greater than a thermal conductivity of the first segment and a thermal conductivity of the third segment. While the percent of increase of the thermal conductivity of the second segment relative to the thermal conductivity of the first segment and the thermal conductivity of the third segment can vary, the thermal conductivity of the second segment is preferably at least five percent, fifteen percent, twenty-five percent, or thirty-five percent greater than the thermal conductivity of the first segment and the thermal conductivity of the third segment.

In disposing the second segment of the tube in the labyrinthine channel, the second segment may have a polygonal cross-section, the first segment has a circular cross-section, and the third segment has a circular cross-section.

In disposing the second segment of the tube in the labyrinthine channel, a cross-section of the labyrinthine channel of the breastmilk warmer receives a cross-section of the second segment of the tube.

### BRIEF DESCRIPTION OF THE DRAWINGS

These and other features and advantages of the various examples disclosed herein will be better understood with respect to the following descriptions and drawings, in which:

FIG. 1 is a perspective view of an exemplary feeding tube constructed in accordance with the present disclosure;

FIG. 2A is a cross-section of the feeding tube of FIG. 1, taken along the line 2A-2A of FIG. 1;

FIG. 2B is a cross-section of the feeding tube of FIG. 1, taken along the line 2B-2B of FIG. 1;

FIG. 2C is a cross-section of the feeding tube of FIG. 1, taken along the line 2C-2C of FIG. 1;

FIG. 3 is a top view of an exemplary warming apparatus constructed in accordance with the present disclosure and used with the feeding tube of FIG. 1, illustrating a hinged cover of the warming apparatus in an open position, exposing a labyrinthine channel of the warming apparatus;

FIG. 4 is a partially-exploded cross-section of the warming apparatus of FIG. 3, taken along the line 4-4 of FIG. 3, with a polygonal-shaped cross-section of a segment of tubing near a complementarily-shaped portion of the labyrinthine channel of the warming apparatus;

FIG. 5 is a perspective view of another embodiment of a feeding tube constructed in accordance with the present disclosure;

FIG. 6 is a cross-section of the feeding tube of FIG. 5 taken along the line 6-6 of FIG. 5;

FIG. 7 is a top view of an exemplary warming apparatus constructed in accordance with the present disclosure and used with the feeding tube of FIG. 5, illustrating a hinged cover of the warming apparatus in an open position, exposing a labyrinthine channel of the warming apparatus;

FIG. 8 is a partially-exploded cross-section of the warming apparatus of FIG. 7 along the line 8-8 of FIG. 7, with a rectangular (square)-shaped cross-section of a segment of tubing near a complementarily-shaped portion of the labyrinthine channel of the warming apparatus;

FIG. 9 is a perspective view of another embodiment of a feeding tube constructed in accordance with the present disclosure;

FIG. 10 is a cross-section of the feeding tube of FIG. 9 along the line 10-10 of FIG. 9;

FIG. 11 is a top view of an example warming apparatus constructed in accordance with the present disclosure and used with the feeding tube of FIG. 9, illustrating a hinged cover of the warming apparatus in an open position, exposing a labyrinthine channel of the warming apparatus; and

FIG. 12 is a cross-section of the warming apparatus of FIG. 11 along the line 12-12 of FIG. 11, with a triangular-shaped cross-section of a segment of tubing near a complementarily-shaped portion of the labyrinthine channel of the warming apparatus.

### DETAILED DESCRIPTION

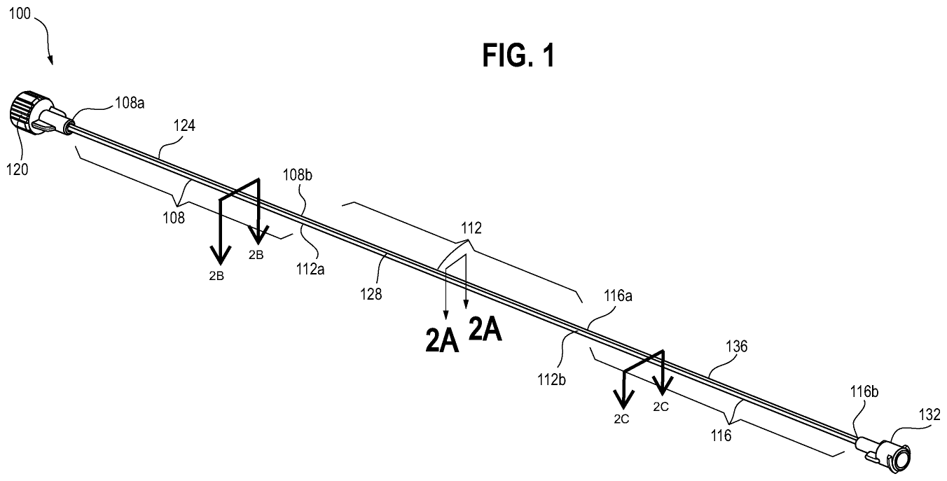

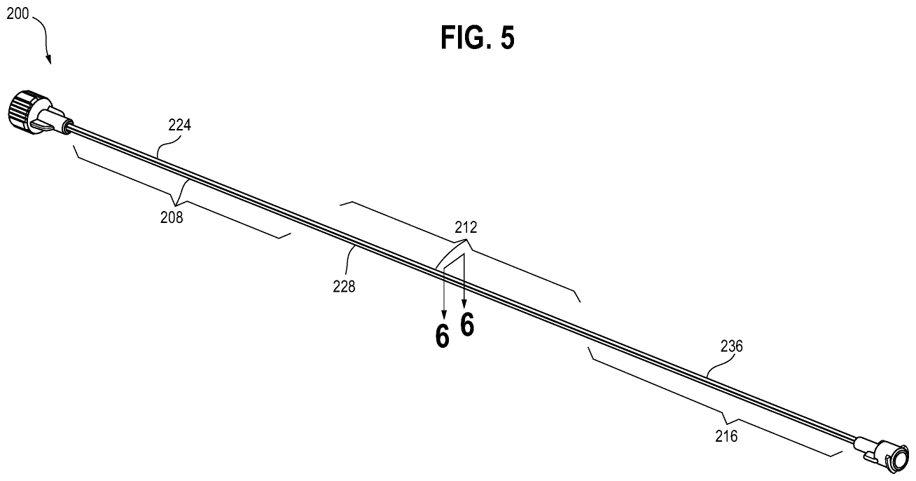

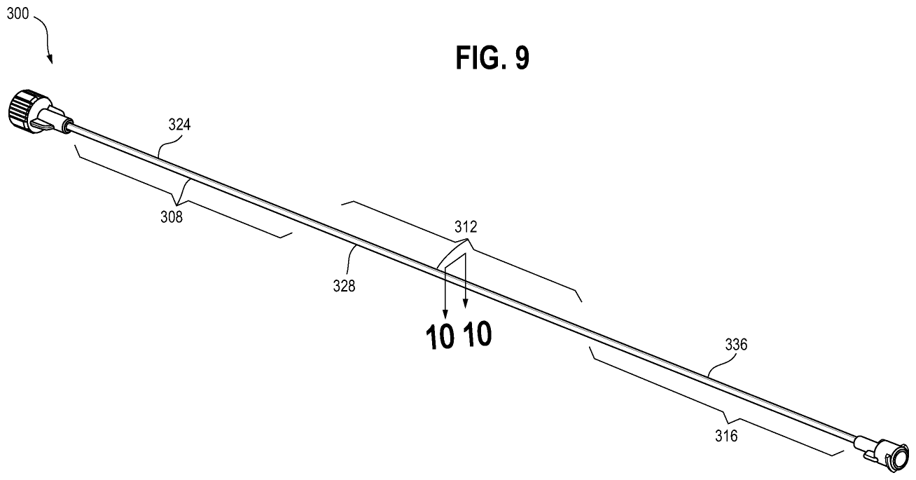

A feeding tube, or tube, as disclosed herein, is configured to be received, at least in part, by a warmer, such that a temperature of a fluid flowing through the tube may be increased when the fluid flows through the portion of the tube that is received by the warmer. A thermal conductivity of the portion of the tube that is received by the warmer may be increased by changing various factors of the portion of the tube. For example, the material, the dimensions, the shape, and several other factors can be changed to influence the thermal conductivity of the tube. In certain instances, factors such as the materials and dimensions might not permissibly or practically be changed due to various external constraints or regulations. As such, the various feeding tubes, or tubes, disclosed herein provide several examples of how to influence the thermal conductivity of the tubes without changing the materials or dimensions of the tube. Rather, the various feeding tubes disclosed herein include differently shaped tubes that influence the thermal conductivity of the portion of the tube received by warmer (though other means of increasing the thermal conductivity of one or more portions of the tube, including the portion of the tube that is received by the warmer, are included in the scope of the present disclosure). For example, FIGS. 1-4 illustrate an embodiment of a feeding tube where the portion of the feeding tube received by the warmer has a hexagonal cross-section. As an alternative example, FIGS. 5-8 illustrate an embodiment of a feeding tube where the portion of the feeding tube received by the warmer has a rectangular (square-like) cross-section. As yet another alternative example, FIGS. 9-12 illustrate an embodiment of a feeding tube where the portion of the feeding tube received by the warmer has a triangular cross-section. In each of the above examples, the portions of the feeding tube that are not received by the warmer have a conventional circular cross-section that has a thermal conductivity that is less than the thermal conductivity of the portion received by the warmer. In other words, the several embodiments disclosed herein illustrate, and the description of the embodiments, set forth various ways as to how, the thermal conductivity of the tube can be increased by changing the shape of the tube received by the warmer.

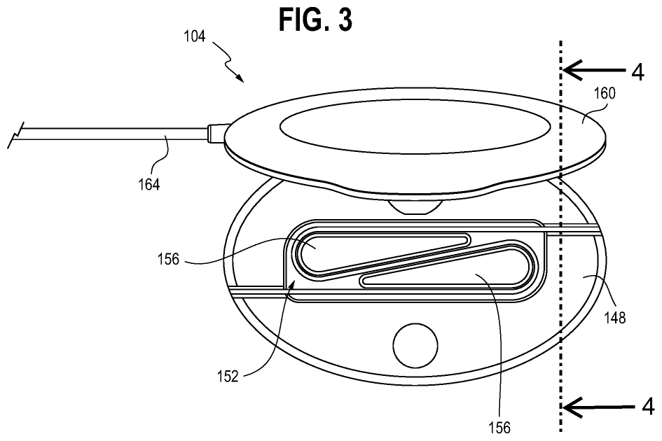

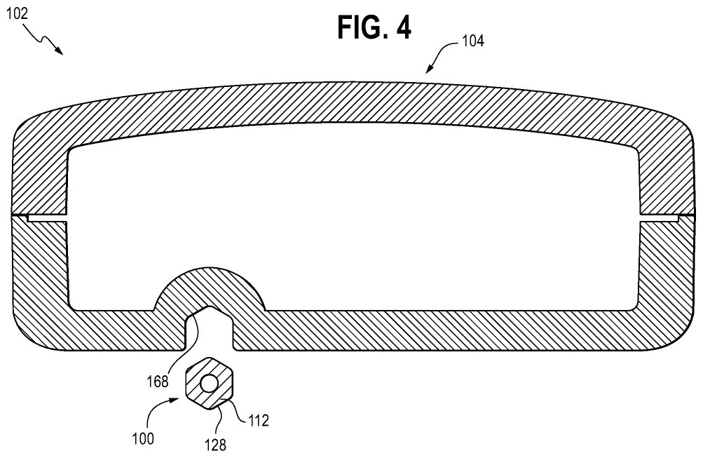

FIG. 1 illustrates a feeding tube, or tube, 100 that is configured to be received, at least in part, by a breastmilk warmer 104 (FIGS. 3 and 4). In the exemplary tube 100 illustrated in FIG. 1, the tube 100 includes a first segment 108 having a first thermal conductivity, a second segment 112 having a second thermal conductivity different from the first thermal conductivity, and a third segment 116 having a third thermal conductivity different from at least one of the first and second thermal conductivities. The first segment 108 is configured to be coupled to a source of fluid and includes a first end 108a and a second end 108b that is opposite of the first end 108a. For example, the source of fluid can be a container with a supply of breastmilk. The first end 108a of the first segment 108 includes a first coupling mechanism 120 that fluidly couples the first segment 108 to the source of fluid. For example, the first coupling mechanism 120 can be a Luer taper and, in particular, a male Luer-lock fitting.

The first segment 108 has a first outer surface 124 that has a first shape. For example, the first outer surface 124 can have a circular shape, which means the first shape has a circular cross-section. In other words, the first segment 108 has a circular cross-section. In other examples, however, the first outer surface 124 can have a rectangular, triangular, trapezoidal, or any other polygonal shape. In either example, the first shape of the first outer surface 124 of the first segment 108 has a first thermal conductivity.

The second segment 112 is fluidly coupled to the first segment and is configured to be disposed in a labyrinthine manner within the breastmilk warmer 104. The second segment 112 includes a first end 112a and a second end 112b that is opposite of the first end 112a. The first end 112a of the second segment 112 is fluidly coupled to the second end 108b of the first segment 108 and the second end 112b of the second segment 112 is fluidly coupled to the third segment 116.

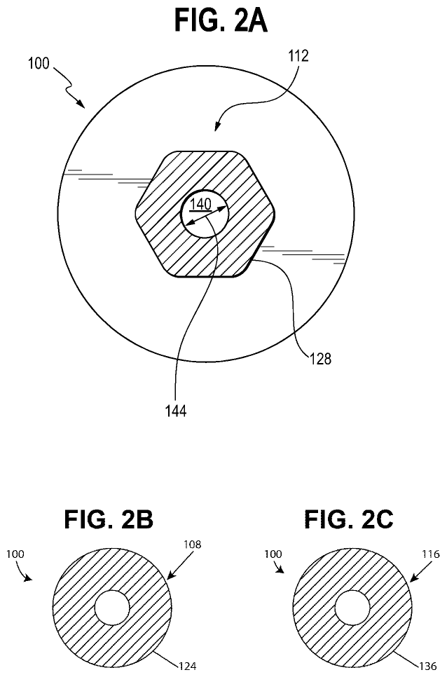

As illustrated in FIG. 2A, the second segment 112 has a second outer surface 128. The outer surface, or the cross-sectional shape of the tube 100, contributes to the heat transfer through the tube 100 to the fluid flowing through the tube 100. As such, modifications to the outer surface, or the cross-sectional shape of the tube 100, can increase or decrease the heat transfer of the tube 100. Importantly, this allows the heat transfer of the tube 100 to increase or decrease without altering other dimensions or sizes of the tube 100, such as its inner (lumen) diameter or length. Accordingly, the second segment 112 has a polygonal cross-section that increases the thermal conductivity of the second segment 112 relative to the thermal conductivity of the first and third segments 108, 116. For example, the second outer surface 128 can have a hexagonal shape, which means the second shape has a hexagonal cross-section. In other words, the second segment 112 has a hexagonal cross-section. Further, based on the second shape of the second segment 112, the second segment 112 has a second thermal conductivity.

The third segment 116 is fluidly coupled to the second segment and is configured to be coupled to a feeding apparatus. The third segment 116 includes a first end 116a and a second end 116b that is opposite of the first end 116a. The second end 116b of the third segment 116 includes a second coupling mechanism 132 that fluidly couples the third segment 116 to the feeding apparatus. For example, the second coupling mechanism 132 can be a Luer tapper and, in particular, a female Luer-lock fitting.

The third segment 116 has a third outer surface 136 that has a third shape. For example, the third outer surface 136 can have a circular shape, which means the third shape has a circular cross-section. In other words, the third segment 116 has a circular cross-section. In other examples, however, the third outer surface 136 can have a rectangular, triangular, trapezoidal, or any other polygonal shape. In either example, the third shape of the third outer surface 136 of the third segment 116 has a third thermal conductivity.

The tube 100 also includes a fluid lumen 140 (FIG. 2A, 2B, 2C) through which fluid can flow from the first segment 108 to the third segment 116. In particular, the fluid lumen 140 has a diameter 144 and extends from the first segment 108 through the second segment 112 and to the third segment 116. As discussed above, the first segment 108 has the first shape, the second segment 112 has the second shape, and the third segment 116 has a third shape. However, despite the shape of the first, second, or third segments, the fluid lumen 140 has a uniform diameter 144 from the first segment to the third segment.

As illustrated best in FIGS. 2A, 2B, and 2C, the second segment 112 has a cross-section that is different from the cross-section of the first segment 108 (FIG. 2B) and the cross-section of the third segment (FIG. 2C). In particular, the second segment 112 has a hexagonal cross-section while the first and third segments 108, 116 have a circular cross-section. While it is not shown in each of the other examples, which will be discussed in further detail below, the same configuration can be found in the example tube 200 of FIGS. 5-8 and the example tube 300 of FIGS. 9-12. However, in other examples, the entire length of the tube 100, 200, 300 can be of a uniform shape that is identical to the shape of the second segment 112, 212, 312. Having a uniform shape throughout the length of the tube 100, 200, 300 may result in a simpler and less costly manufacturing process by not having to create a mold, or other structure, with several different shapes to account for the various shapes.

Turning now to FIGS. 3 and 4, an example breastmilk warming system 102 including a breastmilk warmer 104 and the tube 100 is illustrated. The breastmilk warmer 104 receives a portion of the tube 100 and warms the portion of the tube 100 received. So configured, the breastmilk warmer 104 warms the fluid (e.g., breastmilk) flowing through the portion of the tube 100 that is received by the breastmilk warmer 100. The breastmilk warmer 104 has, in particular, a housing 148, a labyrinthine channel 152 disposed within the housing 148, and a warming assembly 156 that is in communication with the labyrinthine channel 152 and is configured to increase a temperature of the labyrinthine channel 152. The housing 148 has a generally oval shape and retains the labyrinthine channel 152, warming assembly 156, and other electronic components. The housing 148 also includes a cover 160 and a cable 164 that is used to provide power to the warming assembly 156 from a power source (not shown).

The labyrinthine channel 152 is disposed within the housing 148 and receives a portion of the tube 100. As illustrated in FIG. 4, the labyrinthine channel 152 includes a surface 168 that is shaped to receive a portion of the tube 100. In particular, the surface 168 of the labyrinthine channel 152 is shaped to receive the second segment 112 of the tube 100. So configured, the surface 168 of the labyrinthine channel 152 has a hexagonal shape that receives the hexagonal second outer surface 128 of the second segment 108 of the tube 100.

The warming assembly 156 is in communication with the labyrinthine channel 152 such that the warming assembly 156 can increase the temperature of the second segment 112 of the tube 100 received in the labyrinthine channel 152. In particular, the warming assembly 156 forms the labyrinthine channel 152 such that the second segment 112 of the tube 100 wraps around the warming assembly 156. So configured, the second segment 112 of the tube 100 is in contact with the warming assembly 156.

FIGS. 5-8 illustrate another example of a tube 200, a breastmilk warming system 202, and a breastmilk warmer 204 constructed in accordance with the present disclosure. The tube 200 and breastmilk warmer 204 of FIGS. 5-8 are similar to the tube 100 and breastmilk warmer 104 of FIGS. 1-4, except the shape of the tube 200 and the configuration of the breastmilk warmer 204. For example, the tube 200 of FIGS. 5-8 has a portion of the tube that has a rectangular (square-like) cross-section instead of a hexagonal cross-section like the tube 100 of FIGS. 1-4. Thus, for ease of reference, and to the extent possible, the same or similar components of the tube 200 and the breastmilk warmer 204 of FIGS. 5-8 retain the same reference numbers as outlined above with respect to the tube 100 and breastmilk warmer 104 of FIGS. 1-4, although the reference numbers are increased by 100.

Similar to the tube 100 of FIGS. 1, 2, and 4, the tube 200 of FIGS. 5, 6, and 8 includes a first segment 208 having a first thermal conductivity, a second segment 212 having a second thermal conductivity that is different from the first thermal conductivity, and a third segment 216 having a third thermal conductivity that is different from at least one of the first and second thermal conductivities. However, the tube 200 of FIGS. 5, 6, and 8 includes second segment 212 that has a different shape, namely rectangular (e.g., square), from the hexagonal-shaped second segment 112 of the tube 100 of FIGS. 1, 2, and 4.

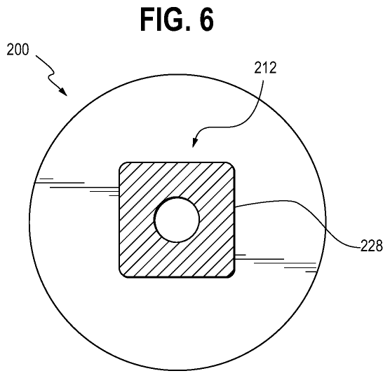

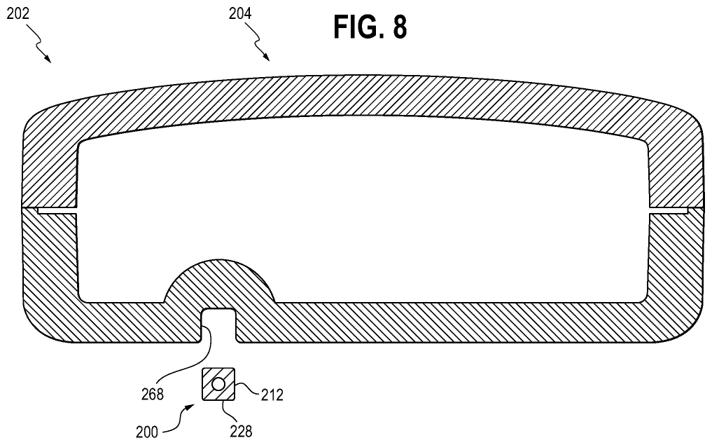

The second segment 212 of the tube 200 has a second outer surface 228. The outer surface, or the shape of the tube 200, determines the heat transfer through the tube 200 to the fluid flowing through the tube 200. So, the outer surface, or the shape of the tube 200, can be changed to increase or decrease the heat transfer of the tube 200. Importantly, this allows the heat transfer of the tube 200 to increase or decrease without altering other dimensions or sizes of the tube 200, such as its inner (lumen) diameter or length. Accordingly, the second segment 212 has a polygonal cross-section that increases a thermal conductivity of the second segment 212 relative to the thermal conductivity of the first and third segments 208, 216. So, as illustrated in FIGS. 6 and 8, the second outer surface 228 can have rectangular shape, which means the second shape has a rectangular cross-section. Further, based on the second shape of the second segment 212, the second segment 212 has a second thermal conductivity.

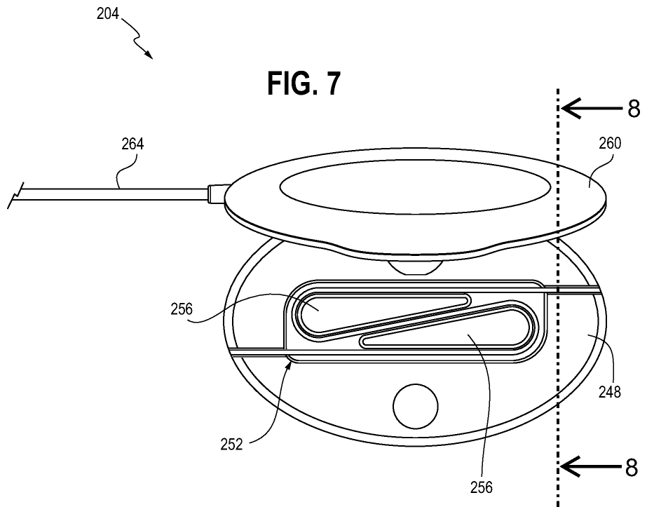

Further, similar to the breastmilk warmer 104 of FIGS. 3 and 4, the breastmilk warmer 204 of FIGS. 7 and 8 includes a housing 248 having a cover 160 and a cable 164, a labyrinthine channel 252 disposed within the housing 248, and a warming assembly 256 that is in communication with the labyrinthine channel 252 and is configured to increase a temperature of the labyrinthine channel 252. However, unlike the labyrinthine channel 152 of the breastmilk warmer 104 of FIGS. 3 and 4, the labyrinthine channel 252 of the breastmilk warmer 204 of FIGS. 7 and 8 has a different shape.

The labyrinthine channel 252 is disposed within the housing 248 and receives a portion of the tube 200. As illustrated in FIG. 8, the labyrinthine channel 252 includes a surface 268 that is shaped to receive a portion of the tube 200. In particular, the surface 268 of the labyrinthine channel 252 is shaped to receive the second segment 212 of the tube 200. So configured, the surface 268 of the labyrinthine channel 252 has a rectangular shape that receives the rectangular second outer surface 228 of the second segment 212 of the tube 200.

FIGS. 9-12 illustrate another example of a tube 300, a breastmilk warming system 302, and a breastmilk warmer 304 constructed in accordance with the present disclosure. The tube 300 and breastmilk warmer 304 of FIGS. 9-12 are similar to the tube 100 and breastmilk warmer 104 of FIGS. 1-4, except the shape of the tube 300 and the configuration of the breastmilk warmer 304. For example, the tube 300 of FIGS. 9-12 has a portion of the tube that has a triangular cross-section instead of a rectangular cross-section like the tube 200 of FIGS. 5-8. Thus, for ease of reference, and to the extent possible, the same or similar components of the tube 300 and the breastmilk warmer 304 of FIGS. 9-12 will retain the same reference numbers as outlined above with respect to the tube 100 and breastmilk warmer 104 of FIGS. 1-4, although the reference numbers will be increased by 200.

Similar to the tube 100 of FIGS. 1, 2, and 4, the tube 300 of FIGS. 9, 10, and 12 includes a first segment 308 having a first thermal conductivity, a second segment 312 having a second thermal conductivity that is different from the first thermal conductivity, and a third segment 316 having a third thermal conductivity that is different from at least one of the first and second thermal conductivities. However, unlike the tube 100 of FIGS. 1, 2, and 4, the tube 300 of FIGS. 9, 10, and 12 includes second segment 312 that has a different shape from the second segment 112 of the tube 100 of FIGS. 1, 2, and 4.

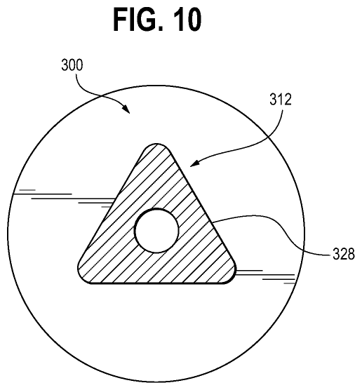

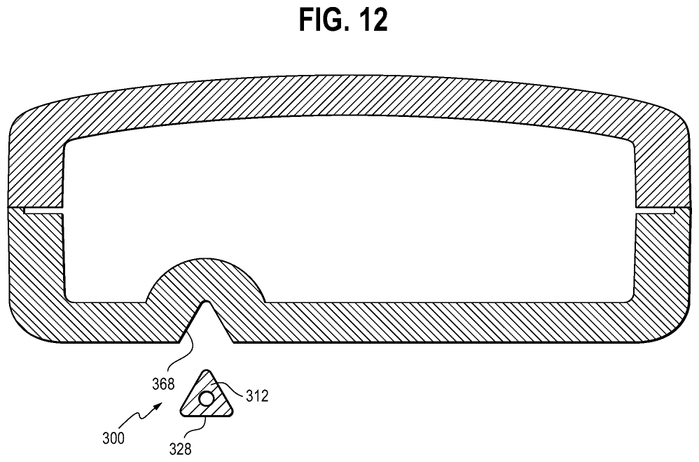

The second segment 312 of the tube 300 has a second outer surface 328. The outer surface, or the shape of the tube 300, determines the heat transfer through the tube 300 to the fluid flowing through the tube 300. So, the outer surface, or the shape of the tube 300, can be changed to increase or decrease the heat transfer of the tube 300. Importantly, this allows the heat transfer of the tube 300 to increase or decrease without changing other dimensions or sizes of the tube 300. Accordingly, the second segment 312 has a triangular cross-section that increases a thermal conductivity of the second segment 313 relative to the thermal conductivity of the first and third segments 308, 316. So, as illustrated in FIGS. 10 and 12, the second outer surface 328 can have triangular shape, which means the second shape has a triangular cross-section. Further, based on the second shape of the second segment 312, the second segment 312 has a second thermal conductivity.

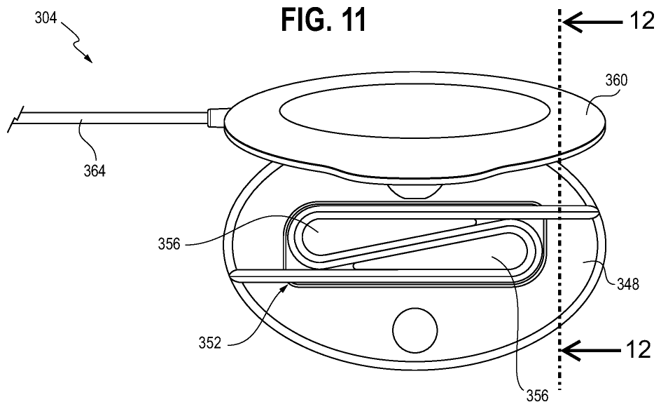

Further, similar to the breastmilk warmer 104 of FIGS. 3 and 4, the breastmilk warmer 304 of FIGS. 11 and 12 includes a housing 348 having a cover 360 and a cable 364, a labyrinthine channel 352 disposed within the housing 348, and a warming assembly 356 that is in communication with the labyrinthine channel 352 and is configured to increase a temperature of the labyrinthine channel 352. However, unlike the labyrinthine channel 152 of the breastmilk warmer 104 of FIGS. 3 and 4, the labyrinthine channel 352 of the breastmilk warmer 304 of FIGS. 11 and 12 has a different shape.

The labyrinthine channel 352 is disposed within the housing 348 and receives a portion of the tube 300. As illustrated in FIG. 12, the labyrinthine channel 352 includes a surface 368 that is shaped to receive a portion of the tube 300. In particular, the surface 368 of the labyrinthine channel 352 is shaped to receive the second segment 312 of the tube 300. So configured, the surface 368 of the labyrinthine channel 352 has a triangular shape that receives the triangular second outer surface 328 of the second segment 312 of the tube 300.

As briefly mentioned above, the shape of the tube 100, 200, 300 and, in particular the shape of the second segment 112, 212, 312 received by the labyrinthine channel 152, 252, 353 of the breastmilk warmer 104, 204, 304, influences the heat transfer rate of the tube 100, 200, 300. In particular, heat transfer rate is given by the following equation (“the Heat Transfer Equation”):

$$\frac{Q}{t} = \frac{kA ( {T}_{2} - {T}_{1} )}{d} .$$

### In the Heat Transfer Equation, the

$$\frac{Q}{t}$$

is the neat transfer rate, k is the thermal conductivity of the material, A is the surface area of the tubing 100, 200, 300, T₂−T₁ is the different in temperature in the system, and d is the thickness of the material.

When calculating the heat transfer rate for the hexagonal cross-section of the second segment 112 of the tube 100 of FIGS. 2 and 4, the heat transfer rate for the triangular cross-section of the second segment 212 of the tube 200 of FIGS. 6 and 8, or the heat transfer rate for the rectangular cross-section of the second segment 313 of the tube 300 of FIGS. 10 and 12, certain variables remain constant while other variables change based on the cross-section of the second segment 112, 212, 313 of the tube 100, 200, 300. For example, the difference in temperature, T₂−T₁ remains constant between the various shapes, the thermal conductivity of the material, k, remains constant between the various shapes (provided the same material is used for all shapes), and the thickness of the material, d, remains constant between the various shapes. Accordingly, the area, or the surface area, A, of the second segment 112, 212, 313 varies depending on the shape of the second outer surface 128, 228, 328.

To determine the surface area of the shape of the second outer surface 128, 228, 328, the following equation can be used: A=l*p, where l is the length of the tube and p is the perimeter. However, as an example, the length of the tube 100 of FIG. 1, the length of the tube 200 of FIG. 5, and the length of the tube 300 of FIG. 9 are the same. As such, the perimeter of the second segment 112, 212, 312 will vary and, ultimately, determine the thermal conductivity of the tube 100, 200, 300. So, as the perimeter increases the heat transfer rate (or thermal conductivity) of the second segment 112, 212, 312 increases. The area of the various shapes of the tubing 100, 200, 300 can be calculates using the follow equations: (1) for the area of a circle, A=πr²; (2) for the area of a hexagon,

$$A = \frac{3 \sqrt{3}}{2} {a}^{2} ,$$

where a is a side length and

$$a = {3}^{1 / 4} \sqrt{2 \frac{A}{9}} ;$$

(3) the area of a square, A=a²; a=\sqrt{A}; and (4) for the area of a triangle,

$$A = \frac{\sqrt{3}}{4} {a}^{2} ; a = \frac{2}{3} {3}^{3 / 4} \sqrt{A} .$$

By way of example only, the perimeter and the heat transfer rate were calculated for a circular cross-section, a hexagonal cross-section, a rectangular cross-section, and a triangular cross-section.

TABLE 1
Percentage of Increased Heat Transfer
Rate for Variously Shaped Tubes

| | Radi- | | | Improved Heat Transfer Rate |
|---|---|---|---|---|
| Shape | us | Area | Perimeter | (relative to a circular shape) |
| Circle | 1 | 3.141593 | 6.283185 | 0% |
| Hexagon | N/A | 3.141593 | 6.597817 | 10% |
| Square | N/A | 3.141593 | 7.089815 | 33% |
| Triangle | N/A | 3.141593 | 8.080642 | 35% |

As can be seen in Table 1 above, a hexagonal cross-section has a heat transfer rate that is 10% greater than the heat transfer rate of a circular cross-section, a square cross-section has a heat transfer rate that is 33% greater than the heat transfer rate of the circular cross-section, and a triangular cross-section has a heat transfer rate that is 35% greater than the heat transfer rate of the circular cross-section. So, the second thermal conductivity of the second segment 112 (hexagonal cross-section) of the tube 100 of FIGS. 1-4 is at least 10% greater than the first thermal conductivity of the first segment 108 (circular cross-section) and the third thermal conductivity of the third segment 116 (circular cross-section) of the tube 100 of FIGS. 1-4. In terms of the tube 200 of FIGS. 5-8, the second thermal conductivity of the second segment 212 (rectangular cross-section) of the tube 200 of FIGS. 5-8 is at least 33% greater than the first thermal conductivity of the first segment 108 (circular cross-section) and the third thermal conductivity of the third segment 216 (circular cross-section) of the tube 200 of FIGS. 5-8. In terms of the tube 300 of FIGS. 9-12, the second thermal conductivity of the second segment 312 (triangular cross-section) of the tube 300 of FIGS. 9-12 is at least 33% greater than the first thermal conductivity of the first segment 308 (circular cross-section) and the third thermal conductivity of the third segment 316 (circular cross-section) of the tube 300 of FIGS. 9-12.

The data in Table 1, above, are merely examples of the possible heat transfer rates that can be achieved based on the various shapes of the second segment 112, 212, 312 of the tube 100, 200, 300. In other examples, the second thermal conductivity of the second segment 112, 212, 312 can be at least five percent greater than the first thermal conductivity of the first segment 108, 208, 308 and the third thermal conductivity of the third segment 116, 216, 316. In another example, the second thermal conductivity of the second segment 112, 212, 312 can be at least fifteen percent greater than the first thermal conductivity of the first segment 108, 208, 308 and the third thermal conductivity of the third segment 116, 216, 316. In yet other examples, the second thermal conductivity of the second segment 112, 212, 312 can be at least twenty-five percent greater than the first thermal conductivity of the first segment 108, 208, 308 and the third thermal conductivity of the third segment 116, 216, 316. Lastly, in some examples, the second thermal conductivity of the second segment 112, 212, 312 can be at least thirty-five percent greater than the first thermal conductivity of the first segment 108, 208, 308 and the third thermal conductivity of the third segment 116, 216, 316.

In view of the foregoing, it is understood that the tube used in the breastmilk warming system 102, 202, 302 may employ the following method for warming breastmilk. For example, the method can include providing a breastmilk warmer 104, 204, 304 including a housing 148, 248, 348, a labyrinthine channel 152, 252, 352 disposed within the housing 148, 248, 438, and a warming assembly 156, 256, 356 in communication with and configured to increase a temperature of the labyrinthine channel 152, 252, 352. The method can include coupling a first segment 108, 208, 308 of the tube 100, 200, 300 to a source of fluid. The tube 100, 200, 300 has a second segment 112, 212, 312 fluidly coupled to the first segment 108, 208, 308 and a third segment 116, 216, 316 fluidly coupled to the second segment 112, 212, 312. The method can also include disposing the second segment 112, 212, 312 of the tube 100, 200, 300 in the labyrinthine channel 152, 252, 352 of the breastmilk warmer 104, 204, 304. Once the second segment 112, 212, 312 is disposed in the labyrinthine channel 152, 252, 352, the method can include activating the warming assembly 156, 256, 356 thereby increasing a temperature of the labyrinthine channel 152, 252, 352 such that a temperature of a fluid from the source of fluid flowing through the second segment 112, 212, 312 increases.

Further, in disposing the second segment 112, 212, 312 of the tube 100, 200, 300 in the labyrinthine channel 152, 252, 352, the second segment 112, 212, 312 has a thermal conductivity at least five percent greater than a thermal conductivity of the first segment 108, 208, 208 and a thermal conductivity of the third segment 116, 216, 316. In another example, in disposing the second segment 112, 212, 312 of the tube 100, 200, 300 in the labyrinthine channel 152, 252, 352, the second segment 112, 212, 312 has a thermal conductivity at least fifteen percent greater than a thermal conductivity of the first segment 108, 208, 308 and a thermal conductivity of the third segment 116, 216, 316. In other examples, in disposing the second segment 112, 212, 312 of the tube 100, 200, 300 in the labyrinthine channel 152, 252, 352, the second segment 112, 212, 312 has a thermal conductivity at least twenty-five percent greater than a thermal conductivity of the first segment 108, 208, 308 and a thermal conductivity of the third segment 116, 216, 316. In yet other examples, in disposing the second segment 112, 212, 312 of the tube 100, 200, 300 in the labyrinthine channel 152, 252, 352, the second segment 112, 212, 312 has a thermal conductivity at least thirty-five percent greater than a thermal conductivity of the first segment 108, 208, 308 and a thermal conductivity of the third segment 116, 216, 316.

In some examples, in disposing the second segment 112, 212, 312 of the tube 100, 200, 300 in the labyrinthine channel 152, 252, 352, the second segment 112, 212, 312 can include a polygonal cross-section, the first segment 108, 208, 308 has a circular cross-section, and the third segment 116, 216, 316 has a circular cross-section. In other examples, in disposing the second segment 112, 212, 316 of the tube 100, 200, 300 in the labyrinthine channel 152, 252, 352, a cross-section of the labyrinthine channel 152, 252, 352 of the breastmilk warmer 104, 204, 304 receives a cross-section of the second segment 112, 212, 312 of the tube 100, 200, 300.

In view of the foregoing, it is understood that the tube 100, 200, 300 used in the breastmilk warming system 102, 202, 302 may employ the following method of assembling the breastmilk warmer 104, 204, 304. For example, the method can include providing the tube 100, 200, 300 and the breastmilk warmer 104, 204, 304 having a housing 148, 248, 348, a labyrinthine channel 152, 252, 352 disposed within the housing 148, 248, 348, and a warming assembly 156, 256, 356 in communication with the labyrinthine channel 152, 252, 352 and configured to increase a temperature of the labyrinthine channel 152, 252, 352; coupling a first segment 108, 208, 308 of the tube 100, 200, 300 to a source of fluid; coupling a second segment 112, 212, 312 of the tube 100, 200, 300 to the first segment 108, 208, 308 of the tube 100, 200, 300, the second segment 112, 212, 312 having a second thermal conductivity; coupling a third segment 116, 216, 326 of the tube 100, 200, 300 to the second segment 112, 212, 312, the third segment 116, 216, 326 configured to be coupled to a feeding apparatus; and disposing the second segment 112, 212, 312 of the tube 100, 200, 300 in the labyrinthine channel 152, 252, 352 of the breastmilk warmer 104, 204, 304.

Further, in disposing the second segment 112, 212, 312 of the tube 100, 200, 300 in the labyrinthine channel 152, 252, 352, the second segment 112, 212, 312 has a thermal conductivity at least five percent greater than a thermal conductivity of the first segment 108, 208, 308 and a thermal conductivity of the third segment 116, 216, 316. In another example, in disposing the second segment 112, 212, 312 of the tube 100, 200, 300 in the labyrinthine channel 152, 252, 352, the second segment 112, 212, 312 has a thermal conductivity at least fifteen percent greater than a thermal conductivity of the first segment 108, 208, 308 and a thermal conductivity of the third segment 116, 216, 316. In other examples, in disposing the second segment 112, 212, 312 of the tube 100, 200, 300 in the labyrinthine channel 152, 252, 352, the second segment 112, 212, 312 has a thermal conductivity at least twenty-five percent greater than a thermal conductivity of the first segment 108, 208, 308 and a thermal conductivity of the third segment 116, 216, 316. In yet another example, in disposing the second segment 112, 212, 312 of the tube 100, 200, 300 in the labyrinthine channel 152, 252, 352, the second segment 112, 212, 312 has a thermal conductivity at least thirty-five percent greater than a thermal conductivity of the first segment 108, 208, 308 and a thermal conductivity of the third segment 116, 216, 316.

In some examples, in disposing the second segment 112, 212, 312 of the tube 100, 200, 300 in the labyrinthine channel 152, 252, 352, the second segment 112, 212, 312 can include a polygonal cross-section, the first segment 108, 208, 308 can include a circular cross-section, and the third segment 116, 216, 316 can include a circular cross-section. In other examples, in disposing the second segment 112, 212, 312 of the tube 100, 200, 300 in the labyrinthine channel 152, 252, 352, a cross-section of the labyrinthine channel 152, 252, 352 of the breastmilk warmer 104, 204, 304 can receive a cross-section of the second segment 112, 212, 312 of the tube 100, 200, 300.

The various example tubes 100, 200, 300 illustrated in FIGS. 1-12 depict the second segment 112, 212, 312 as having different shapes to influence the thermal conductivity of the second segment 112, 212, 312 relative to the first segment 108, 208, 308 and the third segment 116, 216, 316. However, it is recognized that characteristics other than the shape of the second segment 112, 212, 312 can be manipulated to provide the second segment 112, 212, 312 with a different thermal conductivity than the first segment 108, 208, 308 and the third segment 116, 216, 316. For example, use of different materials, or embedding metal elements in the walls of the tubing along the second segment 112, 212, 312, can influence the thermal conductivity of the second segment 112, 212, 312 relative to the first segment 108, 208, 308 and the third segment 116, 216, 316.

While various examples have been described above, this disclosure is not intended to be limited thereto. Variations can be made to the disclosed examples that are still within the scope of the appended claims.

1. A tube configured to be received, at least in part, by a breastmilk warmer, the tube comprising:
a first segment having a first thermal conductivity, the first segment configured to be coupled to a source of fluid;
a second segment having a second thermal conductivity different from the first thermal conductivity, the second segment fluidly coupled to the first segment and configured to be disposed in a labyrinthine manner within a breastmilk warmer;
a third segment having a third thermal conductivity different from at least one of the first and second thermal conductivities, the third segment fluidly coupled to the second segment and configured to be coupled to a feeding apparatus; and
a fluid lumen extending from the first segment through the second segment and to the third segment, the fluid lumen having a uniform diameter from the first segment to the third segment.

2. The tube of claim 1, wherein the second thermal conductivity of the second segment is at least five percent greater than the first thermal conductivity of the first segment and the third thermal conductivity of the third segment.

3. The tube of claim 1, wherein the second thermal conductivity of the second segment is at least fifteen percent greater than the first thermal conductivity of the first segment and the third thermal conductivity of the third segment.

4. The tube of claim 1, wherein the second thermal conductivity of the second segment is at least twenty-five percent greater than the first thermal conductivity of the first segment and the third thermal conductivity of the third segment.

5. The tube of claim 1, wherein the second thermal conductivity of the second segment is at least thirty-five percent greater than the first thermal conductivity of the first segment and the third thermal conductivity of the third segment.

6. The tube of claim 1, wherein the first segment has a circular cross-section, the second segment has a polygonal cross-section, and the third segment has a circular cross-section.

7. The tube of claim 1, wherein a cross-section of the first segment has a first shape, a cross-section of the second segment has a second shape, and a cross-section of the third segment has a third shape, the second shape being different than at least one of the first shape and the third shape.

8. The tube of claim 7, wherein the second shape is a triangle and at least one of the first shape and the third shape is a circle.

9. The tube of claim 7, wherein the second shape is a square and at least one of the first shape and the third shape is a circle.

10. The tube of claim 7, wherein the second shape is a hexagon and at least one of the first shape and the third shape is a circle.

11. A tube configured to be received, at least in part, by a breastmilk warmer, the tube comprising:
a first segment having a first outer surface having a first shape, the first segment configured to be coupled to a source of fluid;
a second segment having a second outer surface having a second shape, the second segment configured to be disposed in a labyrinthine manner within a breastmilk warmer;
a third segment fluidly coupled to the second segment and configured to be coupled to a feeding apparatus; and
a fluid lumen extending from the first segment through the second segment and to the third segment, the fluid lumen having a uniform diameter from the first segment to the third segment,
wherein, the second shape increases a thermal conductivity of the second segment relative to a thermal conductivity of the first segment and a thermal conductivity of the third segment.

12. The tube of claim 11, wherein the second shape of the second segment increases the thermal conductivity of the second segment by at least five percent greater than the thermal conductivity of the first segment and the thermal conductivity of the third segment.

13. The tube of claim 11, wherein the second shape of the second segment increases the thermal conductivity of the second segment by at least ten percent greater than the thermal conductivity of the first segment and the thermal conductivity of the third segment.

14. The tube of claim 11, wherein the second shape of the second segment increases the thermal conductivity of the second segment by at least twenty percent greater than the thermal conductivity of the first segment and the thermal conductivity of the third segment.

15. The tube of claim 11, wherein the second shape of the second segment increases the thermal conductivity of the second segment by at least thirty-five percent greater than the thermal conductivity of the first segment and the thermal conductivity of the third segment.

16. The tube of claim 11, wherein the first outer surface has a circular cross-section, the second outer surface has a polygonal cross-section, and the third segment has a third outer surface that has a circular cross-section.

17. The tube of claim 11, wherein the second outer surface is a triangle and at least one of the first outer surface and a third outer surface of the third segment is a circle.

18. The tube of claim 11, wherein the second outer surface is a square and at least one of the first outer surface and a third outer surface of the third segment is a circle.

19. The tube of claim 11, wherein the second outer surface is a hexagon and at least one of the first outer surface and a third outer surface of the third segment is a circle.

20. A breastmilk warming system, comprising:
a breastmilk warmer, comprising:
a housing;
a labyrinthine channel disposed within the housing; and
a warming assembly in communication with the labyrinthine channel and configured to increase a temperature of the labyrinthine channel; and
a tube configured to be coupled to a source of fluid and a feeding apparatus, the tube comprising:
a first segment having a first thermal conductivity;
a second segment having a second thermal conductivity different from the first thermal conductivity, the second segment fluidly coupled to the first segment and received in the labyrinthine channel of the breastmilk warmer;
a third segment having a third thermal conductivity different from at least one of the first and second thermal conductivities, the third segment fluidly coupled to the second segment; and
a fluid lumen extending from the first segment through the second segment and to the third segment, the fluid lumen having a uniform diameter from the first segment to the third segment.

21. The breastmilk warming system of claim 20, wherein the second thermal conductivity of the second segment is at least five percent greater than the first thermal conductivity of the first segment and the third thermal conductivity of the third segment.

22. The breastmilk warming system of claim 20, wherein the second thermal conductivity of the second segment is at least fifteen percent greater than the first thermal conductivity of the first segment and the third thermal conductivity of the third segment.

23. The breastmilk warming system of claim 20, wherein the second thermal conductivity of the second segment is at least twenty-five percent greater than the first thermal conductivity of the first segment and the third thermal conductivity of the third segment.

24. The breastmilk warming system of claim 20, wherein the second thermal conductivity of the second segment is at least thirty-five percent greater than the first thermal conductivity of the first segment and the third thermal conductivity of the third segment.

25. The breastmilk warming system of claim 20, wherein the first segment has a circular cross-section, the second segment has a polygonal cross-section, and the third segment has a circular cross-section.

26. The breastmilk warming system of claim 25, wherein the labyrinthine channel has a polygonal cross-section that is substantially similar to the polygonal cross-section of the second segment.

27. The breastmilk warming system of claim 20, wherein a first cross-section of the first segment has a first shape, a second cross-section of the second segment has a second shape, and a third cross-section of the third segment has a third shape, the second shape being different than at least one of the first shape and the third shape.

28. The breastmilk warming system of claim 27, wherein the second shape of the second cross-section is a triangle and at least one of the first shape of the first cross-section and the third shape of the third cross-section is a circle.

29. The breastmilk warming system of claim 28, wherein the labyrinthine channel of the breastmilk warmer has a triangular cross-section that receives the second cross-section of the second shape of the second segment.

30. The breastmilk warming system of claim 27, wherein the second shape of the second cross-section is a square and at least one of the first shape of the first cross-section and the third shape of the third cross-section is a circle.

31. The breastmilk warming system of claim 30, wherein the labyrinthine channel of the breastmilk warmer has a square cross-section that receives the second-cross section of the second shape of the second segment.

32. The breastmilk warming system of claim 27, wherein the second shape of the second cross-section is a hexagon and at least one of the first shape of the first cross-section and the third shape of the third cross-section is a circle.

33. The breastmilk warming system of claim 32, wherein the labyrinthine channel of the breastmilk warmer has a hexagonal cross-section that receives the second-cross section of the second shape of the second segment.

34. A method of warming breastmilk, comprising:
providing a warmer including a housing, a labyrinthine channel disposed within the housing, and a warming assembly in communication with and configured to increase a temperature of the labyrinthine channel;
coupling a first segment of a tube to a source of fluid, the tube having a second segment fluidly coupled to the first segment and a third segment fluidly coupled to the second segment;
disposing the second segment of the tube in the labyrinthine channel of the warmer; and
activating the warming assembly thereby increasing a temperature of the labyrinthine channel such that a temperature of a fluid from the source of fluid flowing through the second segment increases.

35. The method of warming breastmilk of claim 34, and in disposing the second segment of the tube in the labyrinthine channel, the second segment has a thermal conductivity at least five percent greater than a thermal conductivity of the first segment and a thermal conductivity of the third segment.

36. The method of warming breastmilk of claim 34, and in disposing the second segment of the tube in the labyrinthine channel, the second segment has a thermal conductivity at least fifteen percent greater than a thermal conductivity of the first segment and a thermal conductivity of the third segment.

37. The method of warming breastmilk of claim 34, and in disposing the second segment of the tube in the labyrinthine channel, the second segment has a thermal conductivity at least twenty-five percent greater than a thermal conductivity of the first segment and a thermal conductivity of the third segment.

38. The method of warming breastmilk of claim 34, and in disposing the second segment of the tube in the labyrinthine channel, the second segment has a thermal conductivity at least thirty-five percent greater than a thermal conductivity of the first segment and a thermal conductivity of the third segment.

39. The method of warming breastmilk of claim 34, and in disposing the second segment of the tube in the labyrinthine channel, the second segment has a polygonal cross-section, the first segment has a circular cross-section, and the third segment has a circular cross-section.

40. The method of warming breastmilk of claim 34, and in disposing the second segment of the tube in the labyrinthine channel, a cross-section of the labyrinthine channel of the breastmilk warmer receives a cross-section of the second segment of the tube.

41. A method of assembling a breastmilk warming apparatus, comprising:
providing a tube and a breastmilk warmer having a housing, a labyrinthine channel disposed within the housing, and a warming assembly in communication with the labyrinthine channel and configured to increase a temperature of the labyrinthine channel;
coupling a first segment of the tube to a source of fluid;
coupling a second segment of the tube to the first segment of the tube, the second segment having a second thermal conductivity;
coupling a third segment of the tube to the second segment, the third segment configured to be coupled to a feeding apparatus; and
disposing the second segment of the tube in the labyrinthine channel of the breastmilk warmer.

42. The method of assembling the breastmilk warming apparatus of claim 41, and in disposing the second segment of the tube in the labyrinthine channel, the second segment has a thermal conductivity at least five percent greater than a thermal conductivity of the first segment and a thermal conductivity of the third segment.

43. The method of assembling the breastmilk warming apparatus of claim 41, and in disposing the second segment of the tube in the labyrinthine channel, the second segment has a thermal conductivity at least fifteen percent greater than a thermal conductivity of the first segment and a thermal conductivity of the third segment.

44. The method of assembling the breastmilk warming apparatus of claim 41, and in disposing the second segment of the tube in the labyrinthine channel, the second segment has a thermal conductivity at least twenty-five percent greater than a thermal conductivity of the first segment and a thermal conductivity of the third segment.

45. The method of assembling the breastmilk warming apparatus of claim 41, and in disposing the second segment of the tube in the labyrinthine channel, the second segment has a thermal conductivity at least thirty-five percent greater than a thermal conductivity of the first segment and a thermal conductivity of the third segment.

46. The method of assembling the breastmilk warming apparatus of claim 41, and in disposing the second segment of the tube in the labyrinthine channel, the second segment has a polygonal cross-section, the first segment has a circular cross-section, and the third segment has a circular cross-section.

47. The method of assembling the breastmilk warming apparatus of claim 41, and in disposing the second segment of the tube in the labyrinthine channel, a cross-section of the labyrinthine channel of the breastmilk warmer receives a cross-section of the second segment of the tube.
